(19) United States
(12) Patent Application Publication
(10) Pub. No.: US 2026/0114905 A1
(43) Pub. Date: Apr. 30, 2026

(54) BILATERAL ANTERIOR SPINAL SCREWS AND SURGICAL PROCEDURE

(71) Applicant: GLOBUS MEDICAL, INC., AUDUBON, PA (US)

(72) Inventors: Matthew McClelland, Phoenixville, PA (US); John LaColla, West Chester, PA (US)

(21) Appl. No.: 18/928,727

(22) Filed: Oct. 28, 2024

(51) Int. Cl. A61B 17/70

(52) U.S. Cl. CPC A61B 17/7002; A61B 17/7067

(57) ABSTRACT

Devices, systems, and methods configured to treat spinal deformities, such as anterior scoliosis correction. A bilateral anterior system may include a bilateral anterior screw having a threaded shaft with modular connectors at each end. The bilateral anterior screw is configured to extend completely through a vertebral body such that the modular connectors are exposed on both sides of the vertebral body. An ipsilateral construct including a first rod or cord is securable to the modular connector on an ipsilateral side, and a contralateral construct including a second rod or cord is securable to the modular connector on a contralateral side, thereby completing a bilateral stabilization.

### FIELD OF THE INVENTION

The present disclosure relates to surgical devices, and more particularly, to bilateral anterior spinal screws and systems for correcting spinal deformities, such as scoliosis.

### BACKGROUND OF THE INVENTION

For certain scoliosis patient populations with multiple curvatures of the spine, anterior correction techniques may be a beneficial solution to treating these spinal deformities. There is an increasing use of hybrid and bilateral anterior constructs for correcting complex, multi-curve deformities. Both vertebral body tethering (VBT) techniques and standard anterior fusion techniques may be used in these situations. In these cases, the patient's first curve may be instrumented and corrected through an anterior approach from one side of the spine. Once the first curve is addressed, the patient is rotated or flipped and the second curve may be instrumented and corrected through an anterior approach to the opposite side of the spine. This typically leads to one or more levels of the spine, near the thoracolumbar junction, with anterior screws being placed from both sides of that specific level. This leads to, in some cases, up to three or four individual screws needing to be placed within the vertebral body of a single level.

While anterior correction techniques may be beneficial for certain patients, the approach and procedure bring several challenges. Access and visibility are very limited in comparison to a typical posterior approach to the spine. The anatomy of the surrounding area of an anterior procedure also make it a risky approach if proper care is not taken during implantation and correction. Specifically, during these hybrid/bi-lateral anterior procedures, the time needed to safely place individual anterior spinal screws from both sides of the patient's spine can extend the total procedure time compared to a posterior approach. Given the heightened risks of the anterior approach, these procedures place a heavy cognitive load on the surgeon given the extra care needed to successfully perform these anterior techniques, especially when needing to place several screws into the same vertebral body from opposing sides.

As such, there is an unmet clinical need to reduce the risks associated with anterior scoliosis correction and reduce the amount of time it takes to perform complex, bilateral anterior spinal procedures. These needs are present for both vertebral body tethering procedures as well as anterior fusion procedures where both sides of the patient's spine need to be accessed and instrumented.

### SUMMARY OF THE INVENTION

To meet this and other needs, devices, systems, and methods are provided for correcting spinal deformities, such as scoliosis. In particular, a bilateral anterior system may include a bilateral anterior screw having modular connectors at each end, which connect to ipsilateral and contralateral spinal rod or cord constructs. Through an anterior or lateral approach, the bilateral anterior screw may be fully inserted through one or more vertebral bodies such that the modular connectors are exposed on each side of the vertebral body. The bilateral anterior screw may have a protective nose cone or replaceable distal tip, for example, to facilitate ease of entry and precise placement though the bone. After insertion, an ipsilateral construct may be completed on the first modular connector of the bilateral anterior screw. For example, the ipsilateral construct may include a first modular tulip placed on the first modular connector and a first spinal rod or cord secured in the first modular tulip. The patient is then flipped and accessed on the opposite side. A contralateral construct may be completed on the second modular connector of the bilateral anterior screw. The contralateral construct may include, for example, a second modular tulip placed on the second modular connector and a second spinal rod or cord secured in the second modular tulip.

The bilateral anterior approach provides for a customized and modular system for anterior fusion and/or vertebral body tethering correction procedures. The bilateral anterior system may be especially suitable for correcting complex, multi-curve deformities, such as scoliosis, where both sides of the patient's spine need to be accessed and instrumented. The bilateral anterior system may provide a faster, safer, and more reliable way of addressing anterior spinal procedures. The surgery may be performed freehand or with the assistance of robotic and/or navigational systems to further improve the safety and time-saving benefits of the bilateral anterior system.

According to one embodiment, a bilateral anterior system includes a bilateral anterior screw, an ipsilateral construct, and a contralateral construct. The bilateral anterior screw has a threaded shaft with modular connectors at each end. The bilateral anterior screw is configured to extend completely through a vertebral body such that the modular connectors are exposed on both sides of the vertebral body. The ipsilateral construct includes a first rod or cord securable to the modular connector on an ipsilateral side with a first tulip and a first locking cap. The contralateral construct includes a second rod or cord securable to the modular connector on a contralateral side with a second tulip and a second locking cap.

The bilateral anterior system may include one or more of the following features. The modular connectors may include spherical screw heads on both sides. Alternatively, the modular connectors may include a monoaxial tulip on the ipsilateral side and a spherical screw head on the contralateral side. The bilateral anterior screw may include a temporary nose cone configured to cover the modular connector on the contralateral side during insertion. Alternatively, the bilateral anterior screw may include a modular nose cone which is interchangeable with the modular connector on the contralateral side. The bilateral anterior screw may include an outer shaft and inner shaft that is permitted to translate relative to the outer shaft to extend an overall length of the bilateral anterior screw. The outer shaft may define one or more interior threads configured to mate with corresponding threads on a proximal drive portion of the inner shaft, and the proximal drive portion may define a drive recess reachable through an access channel through the modular connector on the ipsilateral side. The first and second tulips may each include a tulip head having two arms defining a slot therebetween for receiving the first or second rod or cord therein, and the first and second locking caps may include threaded set screws configured to secure the first or second rod or cord in the respective tulip heads.

According to one embodiment, a method for treating a spinal deformity of a patient may include one or more of the following steps, in any suitable order:
(1) accessing a vertebral body of a vertebra through an anterior or lateral approach; (2) inserting a bilateral anterior screw completely through the vertebral body such that modular connectors are exposable on an ipsilateral side and a contralateral side of the vertebral body; (3) attaching and securing a first rod or cord onto the modular connector on the ipsilateral side to form an ipsilateral construct; (4) flipping the patient over and accessing the vertebral body on the contralateral side; (5) exposing the modular connector on the contralateral side by: (a) removing a temporary nose cone to reveal the modular connector hidden by the temporary nose cone, or (b) replacing a modular nose cone with the modular connector; and (6) attaching and securing a second rod or cord onto the modular connector on the contralateral side to form a contralateral construct. The bilateral anterior screw may traverse generally laterally across the vertebral body and span an entire width of the vertebral body. The bilateral anterior screw may pass through a first cortical wall on the ipsilateral side, through cancellous bone, and through a second cortical wall on the contralateral side of the vertebral body. Prior to flipping the patient over, the method may also include extending an overall length of the bilateral anterior screw by rotating an inner shaft relative to an outer shaft of the screw. The ipsilateral and contralateral constructs may be extended and secured to additional vertebrae. Prior to accessing the vertebral body, the method may include pre-planning or auto-planning a screw trajectory for the bilateral anterior screw via navigation and/or robotic assistance. The ipsilateral and contralateral constructs may include vertebral body tethering and vertebral body tethering, rod fusion and rod fusion, or a combination of both.

According to one embodiment, a bilateral anterior screw assembly includes a bilateral anterior screw having a threaded shaft with a proximal modular connector at one end and a distal modular connector at an opposite end, and a removable nose cone configured to temporarily cover the distal modular connector for insertion of the bilateral anterior screw into bone. The bilateral anterior screw is configured to extend completely through a vertebral body, and after implantation, the nose cone is removable to expose the distal modular connector. The distal modular connector may define a connector recess with interior threads that mate with a post having corresponding threads within the nose cone. The proximal and distal modular connectors may each include spherical screw heads. The nose cone may define a cavity, which receives the spherical screw head of the distal modular connector, and a tapered outer body that terminates as a blunt distal tip. The nose cone may include one or more threads, which are timed with bone threads along the threaded shaft.

According to one embodiment, a bilateral anterior screw assembly includes a bilateral anterior screw having a threaded shaft with a proximal modular connector at one end and a connector recess at an opposite end. The connector recess is configured to receive a portion of either a modular nose cone or a distal modular connector. The modular nose cone and the distal modular connector may each have a central post with threads configured to mate with interior threads in the connector recess. The modular nose cone may temporarily form a distal tip of the threaded shaft during insertion, and after implantation, the modular nose cone may be replaced with the distal modular connector. The bilateral anterior screw may optionally have an adjustable length.

Also provided are kits including bilateral anterior screws of varying types and sizes, tulip assemblies of varying types, spinal rods and cords, locking caps, k-wires, insertion tools, instruments, and other components for performing the procedure(s).

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a perspective view of a single vertebra with a bilateral anterior spinal system including a bilateral anterior screw extending fully through the vertebral body for securing ipsilateral and contralateral spinal rod or cord constructs according to one embodiment;

FIGS. 2A-2D show perspective, side, and cross-sectional views, respectively, of a bilateral anterior spinal screw having modular screw heads at each end with the distal one being temporarily covered by a nose cone for insertion through the vertebral body according to one embodiment;

FIGS. 3A-3B show side views of exploded and assembled components, respectively, of a bilateral anterior spinal screw having a fixed tulip head at one end and a modular spherical screw head at the other temporarily covered by the nose cone for insertion according to one embodiment;

FIGS. 4A-4C show cross-sectional views of a bilateral anterior screw assembly with a modular nose cone that is removable and replaceable with a modular screw head via an internally threaded connection according to one embodiment;

FIG. 5 shows a cross-sectional view of a bilateral anterior screw with a set screw locking nose cone assembly according to one embodiment;

FIGS. 6A-6B show cross-sectional views of an adjustable bilateral anterior screw assembly having an expandable length and a modular nose cone that is replaceable with a modular screw head according to one embodiment;

FIGS. 7A-7B show cross-sectional views of a small diameter adjustable bilateral anterior screw assembly with expandable lengths and replaceable modular tips according to one embodiment;

FIGS. 8A-8F depict a method of inserting a bilateral anterior screw fully through a vertebral body with a temporary nose cone, completing the ipsilateral rod or cord construct, and after flipping the patient over, removing the temporary nose cone, completing the contralateral rod or cord construct according to one embodiment; and

FIGS. 9A-9B depict examples of freehand and navigated/robotic workflows, respectively, utilizing the dual-headed bilateral anterior screw(s) for anterior spinal correction.

### DETAILED DESCRIPTION OF THE INVENTION

Devices, systems, and methods are configured to treat spinal deformities, such as anterior scoliosis correction. A bilateral anterior system may be used to secure ipsilateral and contralateral spinal rod and/or cord constructs. The bilateral anterior system may include a bilateral anterior screw having modular connectors at each end. The bilateral anterior screw(s) may be completely inserted through one or more vertebral bodies, allowing the modular connectors to protrude laterally from both sides of the vertebral body. The bilateral anterior screw may be equipped with a protective nose cone or a replaceable distal tip to enhance ease of insertion and ensure accurate placement through the bone. Once the bilateral anterior screw is in place, construction on the ipsilateral side may begin, for example, with the attachment of a first modular tulip to the first modular connector. This may be followed by securing a first spinal rod or cord within the first tulip, for example, with a first locking cap. Each vertebral level undergoing correction may receive a bilateral anterior screw, traditional anterior screw(s), or other suitable instrumentation to secure the first spinal rod or cord, thereby finalizing the ipsilateral construct. Once the ipsilateral construct is completed, the patient is repositioned and access is gained from the opposite side to build the contralateral construct. Once the patient is flipped and the temporary nose cone is removed or replaced with the second modular connector, construction on the contralateral side may begin. For example, a second modular tulip may be placed on the second modular connector of the bilateral anterior screw. Next, the second spinal rod or cord is secured within the second modular tulip, for example, with a second locking cap. Each vertebral level undergoing correction may receive a bilateral anterior screw, traditional anterior screw(s), or other suitable instrumentation to secure the second spinal rod or cord, thereby finalizing the contralateral construct, and thus finishing the bilateral stabilization.

Although described herein with reference to anterior spinal correction, it will be appreciated that the devices described herein may be applied to other areas of the spine, other orthopedic locations in the body, and other medical procedures, such as trauma applications. Any of the bilateral anterior screws described herein may be offered in a multitude of styles, sizes, and lengths, helping to ensure optimal patient fit and surgeon preference.

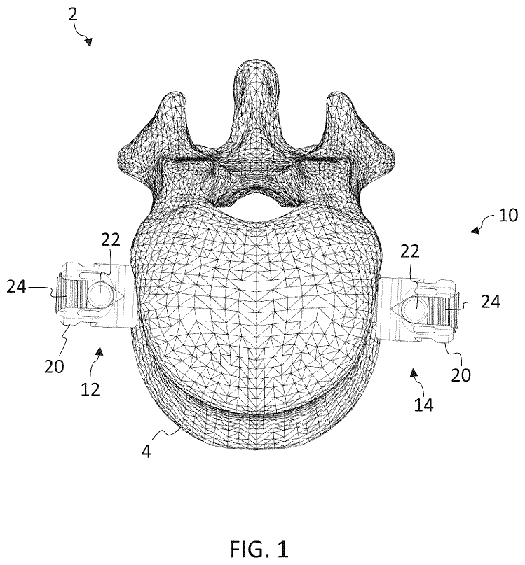

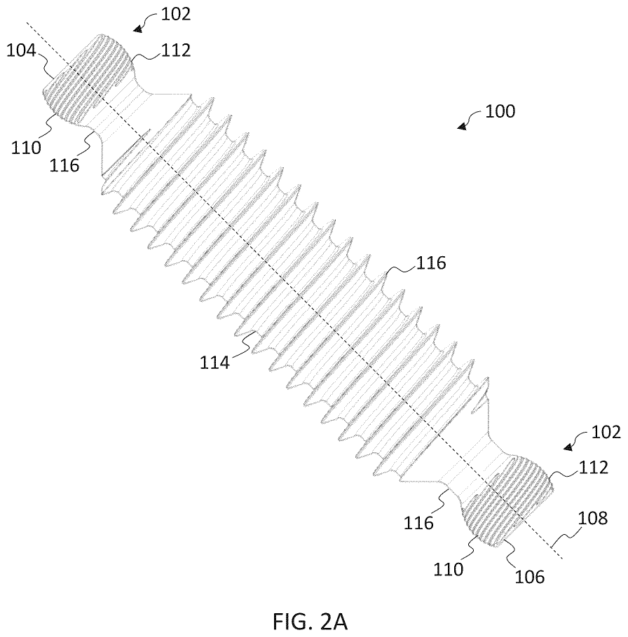

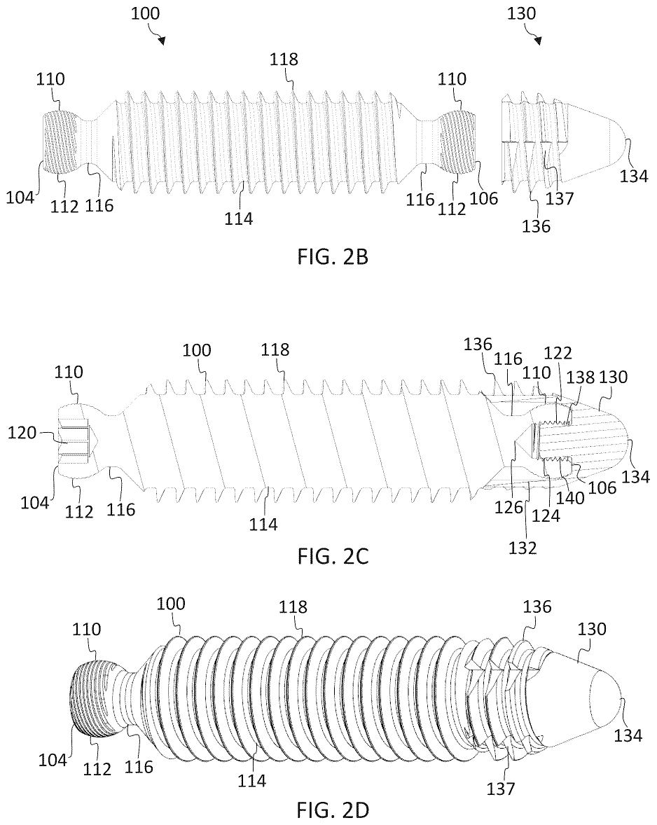

Turning now to the figures, where like reference numbers may refer to like elements, FIG. 1 shows a perspective view of a single vertebra 2 with a bilateral anterior spinal system 10 including an ipsilateral construct 12 (on the entry side) and a contralateral construct 14 (on the exit side) according to one embodiment. The bilateral anterior spinal system 10 may include a bilateral anterior screw, such as screw 100 shown in FIG. 2A, completely inserted through the vertebral body 4. The bilateral anterior screw 100 includes connectors 102 at opposite ends, which protrude on either side of the vertebral body 4. The ipsilateral and contralateral constructs 12, 14 are then coupled to the connectors 102 to complete the bilateral anterior spinal system 10. Although depicted with the ipsilateral construct 12 (entry side of screw 100) on the patient's right side and the contralateral construct 14 (exit side of screw 100) on the patient's left side, it will be appreciated that the ipsilateral and contralateral sides may be reversed if the procedure starts on the opposite side instead.

The vertebra 2 is a crucial component of the vertebral column and plays a fundamental role in providing structural support and protecting the spinal cord while allowing a range of movement. The largest part of the vertebra 2, located anteriorly toward the front of the body, is the vertebral body 4. It is made of mostly cancellous or spongy bone encased in a thin shell of cortical or hard bone, which gives it both strength and resilience. The vertebral bodies 4 are separated from each other by intervertebral discs, which function as shock absorbers and allow for mobility at each vertebral segment. The thoracic spine includes twelve vertebrae 2 (T1 through T12), and the lumbar spine includes the lower end of the spinal column between the last thoracic vertebra (T12) and the first sacral vertebra (S1). FIG. 1 depicts vertebra T12, but it will be appreciated that the bilateral anterior spinal system 10 may apply to any level in the thoracolumbar spine. The bilateral anterior spinal system 10 may be especially suitable for thoracic vertebrae T10 through lumbar vertebrae L2. Although only a single vertebra 2 is shown in FIG. 1, it will be appreciated that the system 10 may also be extended to other vertebral levels and/or combined with traditional anterior screws or other instrumentation to complete the full construct.

The bilateral anterior screw, such as screw 100 shown in FIG. 2A, is inserted through the vertebral body 4 such that the connectors 102 at opposite ends protrude laterally on either side of the vertebral body 4. An anterior or lateral surgical approach may be used to access a lateral portion of the vertebral body 4 on the ipsilateral side and insert the bilateral anterior screw 100. A pathway may be created across the vertebral body 4 such that the bilateral anterior screw 100 extends fully across the vertebral body 4. The bilateral anterior screw 100 may pass through the first cortical wall (on the ipsilateral or entry side), through the cancellous bone, and through the second cortical wall (on the contralateral or exit side) of the vertebral body 4. The bilateral anterior screw 100 may be oriented such that it traverses generally laterally across the vertebral body 4 where the screw 100 spans the entire width of the vertebral body 4. The screw 100 may be guided on a path that allows the screw 100 to pass through or close to the geometric center of the vertebral body 4, for example. This configuration may help to maximize the contact area with bone and distribute the mechanical loads evenly across the vertebral structure, thereby stabilizing and enhancing fixation while reducing the risks of bone stress or screw loosening.

The connectors 102 at each end of the bilateral anterior screw 100 are configured to connect to the respective constructs 12, 14, which are part of the larger spinal fixation system. For example, as described in more detail herein, each connector 102 may include a modular component configured to connect each end of the screw 100 to the respective rod or cord constructs 12, 14. In one embodiment, each connector 102 may include a modular spherical head 110, which is configured to receive a tulip head assembly or tulip 20 thereon. Each tulip 20 may include opposed arms defining a U-shaped channel or slot sized and configured to accept a spinal rod 22 (or cord) therein. The spinal rod 22 (or cord) is configured to be retained within the tulip 20 and may be tensioned intraoperatively to correct the deformity of the spine. The modular nature of each connector 102 gives the flexibility to use these screws 100 for bilateral anterior fusions, bilateral vertebral body tethering (VBT), or a hybrid combination of both.

In a fusion procedure, the spinal rod 22 may be used to stabilize the spine, correct deformities, and maintain proper alignment of the spine. The spinal rod 22 may have an elongated shaft with a generally cylindrical outer body. It will be appreciated that the spinal rod 22 may also have other cross-sectional shapes, such as oval, rectangular, or flattened surfaces. The rod 22 may be made from biocompatible materials, such as titanium, titanium alloys, cobalt-chrome alloys, or stainless steel, that have high tensile strength and can withstand forces and stresses placed on the spine. The rods 22 may range in diameter and length depending on the number of vertebral levels that need to be spanned. The choice of rod type, size, and material may be influenced by the specific surgical goals, the patient's anatomy, and the surgeon's preference. Examples of tulip assemblies and rod constructs are described in more detail, for example, in U.S. Pat. No. 10,368,917, which is incorporated by reference herein in its entirety for all purposes.

In a vertebral body tethering procedure, the spinal rod 22 may be replaced or substituted with a cord or tether. The cord may be a cable, wire, band, flexible or elastic member, for example. The cord may be a single continuous cord extending from one end to the other, but it is also envisioned that more than one cord may be used or a section thereof may be coupled to another cord, rod, or other device, if desired. The cord may be composed of a polymer, such as polyethylene terephthalate (PET), but any suitable biocompatible material may be selected. The cord may be placed under tension to achieve the proper amount of correction to the spine without fusion. The tensioned cord may provide for spinal alignment to maintain stability while allowing growth in skeletally immature patients. Tethering systems are described in more detail, for example, in U.S. Publication No. 2022/0110662, which is incorporated by reference herein in its entirety for all purposes.

Once the spinal rod 22 (or cord) is positioned in the tulip 20, the spinal rod 22 (or cord) may be secured therein with a locking cap 24. For example, each of the arms of the tulip 20 may have interior threaded portions for engaging a threaded locking cap 24, or threaded set screw. The spinal rod 22 (or cord) may be secured in the tulip 20 when the threaded locking cap 24 is threaded downward into the tulip 20. Alternatively, the locking cap and tulip interface may be replaced with a non-threaded interface or another suitable configuration for securing the rod 22 (or cord) in the tulip 20.

It will be appreciated that the tulip assembly 20 may include a tulip head which houses one or more clamping elements, such as a saddle, wedge, clip, or the like. For a rod construct, a polyaxial or uniplanar tulip assembly may be beneficial. For a polyaxial assembly, tightening the locking cap 24 compresses the rod 22 into the tulip 20, thereby restricting angulation and motion of the tulip 20 and forming a rigid construct. For a tethering construct, a monaxial tulip assembly may be useful. Due to the modular nature of the system 10, it will be appreciated that any suitable tulip assembly may be selected including unilateral, monoaxial, polyaxial, fixed, reduction, etc., which offer different degrees of flexibility, stability, and ease of use based on the requirements of the spinal procedure.

The bilateral anterior screws, tulips, locking caps, or components thereof may be comprised of titanium or titanium alloys, stainless steel, cobalt chrome, cobalt-chrome-molybdenum, tungsten carbide, carbon composite, plastic or polymer—such as polyetheretherketone (PEEK), polyethylene, ultra-high molecular weight polyethylene (UHMWPE), combinations of such materials, or any other appropriate material that has sufficient strength to be secured to bone, while also having sufficient biocompatibility to be implanted into the body. The bilateral anterior screws may also undergo a surface treatment or coating, such as hydroxyapatite (HA) coating for better bone purchase. Although the above list of materials includes many typical materials out of which screws and components may be made, it should be understood that any appropriate materials are contemplated.

Turning now to FIGS. 2A-2D, bilateral anterior screw 100 is shown in more detail according to one embodiment. The bilateral anterior screw 100 extends from a proximal end 104 to a distal end 106 along a central screw axis 108. Each end 104, 106 of the screw 100 includes a modular connector 102 configured to couple to the respective rod or cord constructs 12, 14. During insertion into and through bone, the distal connector 102 may be covered or hidden with a temporary nose cone 130. The temporary nose cone 130 shields the distal end 106 of the screw 100 during insertion. After insertion, the temporary nose cone 130 is removed to reveal the distal modular connector 102, thereby allowing for completion of the contralateral rod or cord construct 14.

As shown in this embodiment, each connector 102 may include a modular screw head 110, such as a spherical head, which is configured to receive the tulip 20 thereon. For example, at least a portion of the screw head 110 may be shaped to form a portion of a ball or at least a portion of a sphere. While the screw head 110 may have any general shape, the spherical shape allows for modular connection with different types of tulip assemblies 20. In the case of a polyaxial interface, for example, at least a portion of the screw head 110 has a curved outer surface in order to allow for rotational movement and/or angular adjustment of the tulip 20 with respect to the anterior screw 100. Although a spherical head is exemplified, it will be appreciated that other connections between the screw head 110 and tulip 20 may be selected. In addition, the screw head 110 may have a smooth, threaded, roughened or textured outer surface, or may be otherwise configured to interface with the tulip 20. As shown in this embodiment, the screw head 110 may include one or more threads 112 configured to enhance friction with the tulip assembly 20.

The modular screw heads 110 are provided at the opposing ends 104, 106 of a shaft 114. The shaft 114 may have a generally cylindrical body, typical to a bone screw shank. Each modular screw head 110 may extend from the shaft 114 via a narrowed or reduced diameter neck 116. In this embodiment, a large diameter screw 100 is shown where the necks 116 and screw heads 110 have a reduced diameter relative to the outer diameter of the threaded shaft 114. The necks 116 further include a reduced diameter relative to the outer diameter of the screw heads 110.

The shaft 114 may include one or more exterior bone threads 116 configured to engage bone. The bone threads 116 include external helical ridges that follow a helical path around the periphery of the shaft 114, which are configured for anchoring the screw 100 into bone. Varying bone thread forms may be used. The bone threads 116 may include a single lead with one continuous thread that spirals around the screw's body, dual lead threads, triple lead threads, or other variations, such as variable pitch threads, fluted threads, etc. The threaded shaft 114 may have a number of different features, such as thread pitch, shaft diameter to thread diameter, overall shaft shape, and the like, to optimize bone purchase. Cannulations and fenestrations may also be employed for placement over a guide wire or k-wire and/or for delivery of bone cement. The screws 100 may be made in a variety of diameters and lengths to match the anatomy of the patient. The screws 100 may also optionally undergo a hydroxyapatite (HA) coating, surface treatment, or other additive processes if desired.

As best seen in FIG. 2C, the proximal screw head 110 may define a tool engagement surface or drive recess 120 that can be engaged, for example, by a screw-driving instrument or other device. The drive recess 120 is housed in the top of the screw head 110. The drive recess 120 may have a torx or hexalobe shape, for example, for driving the screw 100 into bone with an appropriate instrument. It will be appreciated that any suitably shaped tool engagement surface may be provided to insert the bone screw 100 into the vertebra 2.

The distal screw head 110 may define a connector recess 122 that can be engaged, for example, with a portion of the temporary nose cone 130. The nose cone 130 is configured to cover and protect the distal screw head 110 when the anterior screw 100 is being inserted into and through bone. In this embodiment, the nose cone 130 is temporarily coupled to the distal screw head 110 with a threaded connection. The connector recess 122 in the distal screw head 110 may include interior threads 124, which mate with corresponding threads 140 within the nose cone 130. The connector recess 122 may be a closed bore or hollow cylindrical space that terminates, for example, at a point 126. The connector recess 122 may extend through the distal screw head 110 and into the neck 116 of the screw 100 along central screw axis 108.

The temporary nose cone 130 may be sized and dimensioned to cover and hide the entire distal screw head 110. The nose cone 130 may define a cavity 132, which receives the distal screw head 110. For example, the nose cone 130 may extend over the distal screw head 110, over the neck 116, and match up to the shaft 114 of the screw 100. The outer diameter of the nose cone 130 may be about the same or slightly smaller than the outer diameter of the shaft 114, thereby providing a seamless extension of the threaded shaft 114. The temporary nose cone 130 includes a tapered outer body that terminates at a distal tip 134. The distal tip 134 may be generally blunt and non-threaded to prevent damage to soft tissue. Alternatively, the distal tip 134 may be pointed or may include cutting edges to aid in starting the screw 100. It will be appreciated that varying tip geometries may be tailored for specific applications. The outer surface of the nose cone 130 includes one or more threads 136, which may match with the threads 118 along the shaft 114. The threads 136 of the nose cone 130 may be timed to the threads 118 of the shaft 114 to facilitate screw insertion. For example, the start and pitch of the threads 136 on the nose cone 130 may match exactly with the threads 118 on the shaft 114. This alignment ensures that when the nose cone 130 is assembled to the shaft 114, the threads 118, 136 seamlessly mesh without cross-threading or misalignment. The nose cone threads 136 may also include cutting flutes 137 or other specialized profiles, for example, to facilitate faster penetration, reduce installation torque, and/or improve precision of the screw installation.

In order to temporarily affix the nose cone 130 to the distal screw head 110, a central post 138 may be present to temporarily connect the nose cone 130 to the screw shaft 114. The central post 138 may be located within cavity 132 such that the post 138 extends proximally along the central screw axis 108. The post 138 may have a cylindrical body with exterior threads 140 configured to mate with the interior threads 124 in the connector recess 122 of the distal screw head 110. The temporary nose cone 130 may be rotated onto the distal end 106 of the screw head 110 such that the distal screw head 110 is fully concealed by the nose cone 130. Although a threaded attachment is exemplified, it will be appreciated that a snap fit, interference fit, snap ring, or other connection mechanism may be used to temporarily connect the nose cone 130 to the distal screw head 110. The presence of the temporary nose cone 130 allows for easy insertion of the screw 100 into and through the vertebral body 4. Once positioned properly, the nose cone 130 may be removed to expose the distal screw head 110 to complete the contralateral construct 14.

In the embodiment shown, the bilateral anterior screw 100 includes a large diameter screw that can be hydroxyapatite (HA) coated for better bone purchase. This screw 100 is specifically configured for the spinal levels in which individual anterior screws are needed on both sides of the vertebral body 4. In order to reduce the total time required to place screws from both sides of the spine, this embodiment of the screw 100 contains modular spherical head 110 on each end 104, 106 of the screw 100. The proximal end 104 of the screw 100 may be the same or similar to a typical modular screw. The spherical head 110 and drive recess 120 are used to rigidly insert the screw 100 into the vertebral body. The driver instrumentation is removed once the screw 100 is fully inserted, and the spherical head 110 remains unobstructed for modular tulip insertion. The ipsilateral construct 12 on this first instrumented side can then be completed once the desired tulip 20 is attached to the spherical head 110. The distal end 106 of the screw 100 has temporary nose cone 130 that shields the screw's second spherical head 110. The nose cone 130 is threaded into the second spherical head 110 and the cone's outer geometry tapers and has threads 136 to mimic the function of the screw shank 114. The nose cone 134 can come with or without cutting flutes 137 depending on surgeon preference. The nose cone outer threads 136 may be timed with the screw's shank threads 118 such that insertion is unaffected and pull out strength and bone purchase are not impacted.

Once the screw 100 is fully inserted through the vertebral body 4, the nose cone 130 has advanced fully through the opposing cortical wall. The nose cone 130 may remain in place until the patient is flipped. This style of screw 100 can be inserted from the first instrumented side of the spine at any level where anterior screws would have otherwise been placed from both sides of the spine. Once the patient is flipped, the opposing contralateral construct 14 can be created. Screws may still need to be inserted into some of the levels for that side's construct, but the levels in which these double-headed screws 100 are placed need no further work done other than removing the nose cone(s) 130 and placing modular tulips 20 on each of the spherical heads 110.

The bilateral design not only reduces the total number of screws needed to be inserted for a given bilateral anterior case, but it also allows for additional customization of the rod and cord constructs 12, 14. In particular, the modular screw heads 110 can allow for not only modular acceptance of polyaxial tulips 20 but also uniplanar or monoaxial variations as well as double headed connectors and other lateral and inline connector options. Examples of some additional connector types are shown, for example, in U.S. Pat. No. 11,612,418 and U.S. Pat. No. 11,723,693, which are incorporated by reference herein in their entireties for all purposes.

The utilization of a larger diameter screw combined with an optional hydroxyapatite (HA) coating provides a number of advantages in spinal surgeries, particularly in enhancing the stability and integration of the screw 100 within the vertebral body 4. The larger diameter screw shank 114 offers a greater surface area in contact with the bone, which allows for better distribution of mechanical loads exerted during movement. Typically, staples or washers may be used to distribute the load over a wider area to prevent the screw from cutting into the bone under load. The larger diameter screw and optional HA coating allows for surgeons to omit the use of staples/washers if they prefer. The larger diameter shank 114 may better support the screw 100 within the vertebral body 4 without needing the staple/washer present to help distribute loads. Similarly, the optional HA coating enhances the biological fixation of the screw 100 by promoting bone growth around and onto the screw's surface. This osteoconductive coating may help to ensure proper osteointegration, further reducing the need for the additional stability of a staple/washer.

The modular design gives the flexibility to use these screws 100 for bilateral anterior fusions, bilateral vertebral body tethering, or a hybrid combination of both. Typically, a monoaxial tulip 20 is most desirable for a tethering construct, where a polyaxial or uniplanar tulip 20 may be more beneficial for an anterior fusion. In some cases, a modular polyaxial tulip 20 may be attached to the spherical modular head 110 and an instrument may lock the polyaxial motion of the tulip 20, creating a monoaxial-acting tulip for any vertebral body tethering construct needs. Additionally, a double-headed tulip connector may be attached to the spherical modular head 110, which allows for a streamlined implantation process for any dual cord or dual rod constructs.

The modularity and double-sided nature of screw 100 reduces the total number of screws needed to be inserted from both sides of the spine. This greatly reduces the total time needed to complete these constructs 12, 14 as well as improves the safety of the procedure when compared to traditional techniques, such as inserting multiple screws into the same vertebral body from both sides of the spine. The customization capabilities of the modular spherical heads 110 allow for a wider range of uses in anterior fusion and vertebral body tethering constructs at the levels of overlapping constructs.

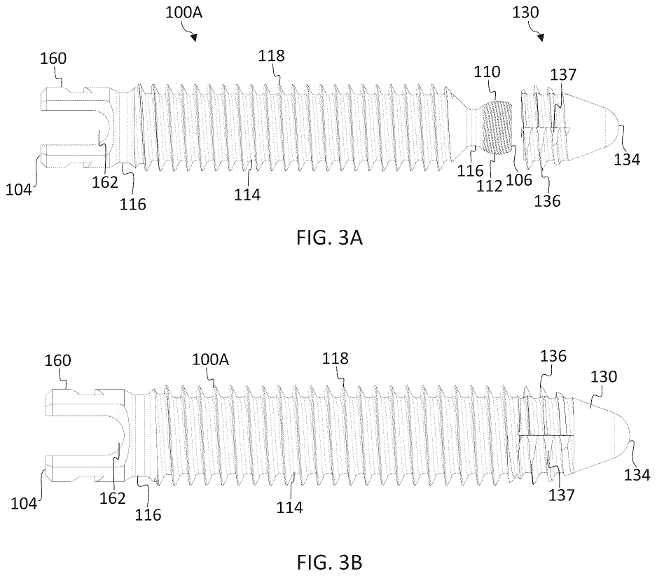

Turning now to FIGS. 3A-3B, a bilateral anterior screw 100A is shown according to another embodiment. This design is very similar to screw 100 except the proximal modular spherical head 110 is replaced by a rigid monoaxial tulip 160. The temporary distal nose cone 130 shields the distal end's spherical head 110 in the same manner as previously explained. Likewise, the temporary nose cone 130 is tapered and can have cutting flute geometry to ease insertion.

The rigid monoaxial tulip 160 may be provided on the proximal end 106 of the shaft 114. Unlike polyaxial tulip heads, which allow for variable angles of attachment relative to the screw shaft, the monoaxial tulip head 160 is fixed in its orientation. In other words, the tulip head 160 and shaft 114 are aligned along a single axis and do not permit angular adjustment after installation. The monoaxial tulip 160 may include opposed arms defining a U-shaped channel or slot 162 sized and configured to accept the spinal rod 22 (or cord) therein. The monoaxial tulip 160 is also configured to receive a locking cap, such as a set screw or locking cap 24, which is used to secure the spinal rod 22 (or cord) into the monoaxial tulip 160 once positioned therein. The monoaxial tulip 160 provides a stable and secure connection between the spinal rod 22 (or cord) and the bilateral anterior screw 100A, which helps to maintain proper alignment of the spine and distribute loads across the spinal segment. A rigid, monoaxial screwdriver may be used to drive the screw 100A into the vertebral body 4. Staples or washers are not needed given the large diameter of the shank 114 and the optional HA coating for improved bone purchase.

This screw design still allows for the customization of tulip selection on the distal screw end 106 but also ensures the rigidity of a true monoaxial screw is still present for any vertebral body tethering or anterior fusion constructs 12, 14. The assembled screw 100A can be inserted through the selected vertebral body 4, leaving the monoaxial tulip 160 on the insertion side of the vertebra 2 and the assembled nose cone 130 on the other. Once the vertebral body tethering or fusion construct 12 is completed on the insertion side, the patient may be flipped, the nose cone 130 removed, and a suitable tulip 20 can be selected and attached for completion of vertebral body tethering or fusion for the contralateral construct 14. This design provides the same benefits as described previously but with the option of having a truly monoaxial screw head 160 on the proximal end 104.

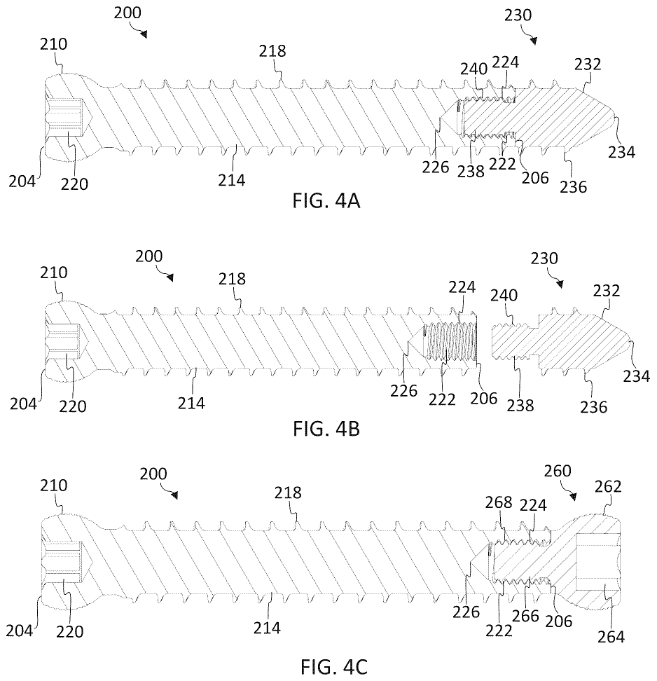

Turning now to FIGS. 4A-4C, a bilateral anterior screw 200 is shown according to another embodiment. The bilateral anterior screw 200 is similar to screw 100 but utilizes a much smaller, internally threaded connection for modular attachment of the distal end 206. The smaller modular connector 222 allows for a smaller nose cone 230 and therefore smaller diameter bilateral screws 200 with features similar to screw 100. These screws 200 may be best suited for smaller stature patients where screws 100, 100A may be too bulky for safe insertion. The smaller stature patient exerts reduced loads on any construct created therefore a smaller, threaded modular connection may be sufficient in maintaining construct integrity.

The bilateral anterior screw 200 extends from a proximal end 204 to a distal end 206. The proximal end 204 includes a modular screw head 210, similar to screw head 110, which is configured to receive the tulip 20 thereon. The screw head 210 may have a spherical or semi-spherical in shape, which allows for motion and articulation of the tulip 20. The screw head 210 defines drive recess 220 for mating with a driver instrument for installing the screw 200. The shaft 214 may have a generally cylindrical body with one or more exterior bone threads 218 configured to engage bone. At the distal end 206, the shaft 214 terminates as a free end. In this embodiment, the shaft 214 defines a connector recess 222 that can be engaged, for example, with a portion of the nose cone 230 or a modular head 260. The connector recess 222 may include interior threads 224, which mate with corresponding threads 240, 268 on the nose cone 230 or modular head 260, respectively. The connector recess 222 may be a closed bore or hollow cylindrical space that terminates, for example, at point 226 within shaft 214.

In this embodiment, the nose cone 230 or modular head 260 may be interchangeably attached to the distal tip 206 of the shaft 214. For insertion into and through the vertebral body 4, the temporary nose cone 230 may be affixed to the screw shaft 214. The temporary nose cone 130 includes a tapered outer body 232 that terminates at a distal tip 234. The outer diameter of the nose cone 230 may be about the same or slightly smaller than the outer diameter of the shaft 214, thereby providing a seamless extension of the threaded shaft 214. The outer surface of the nose cone 230 includes one or more threads 236, which may match with the threads 218 along the shaft 214 of the screw 200. The body 232 includes a central post 238 that extends proximally. The central post 238 may have a cylindrical body with exterior threads 240 configured to mate with the interior threads 224 in the connector recess 222 of the shaft 214. The nose cone 230 may be rotated onto the distal end 206 of the shaft 214 such that the nose cone 230 temporarily forms the distal tip of the screw shaft 214.

After installation and the patient is flipped over, the nose cone 230 may be removed and the modular screw head 260 affixed to the screw shaft 214. Once the nose cone 230 is removed from the contralateral screw tip, the spherical head 260 is attachable to the threaded bore 222 in the screw shaft 214. The modular screw head 260 includes a spherical head portion 262 with a drive recess 264, similar to screw head 210. The central post 266 extends proximally from the head portion 262. The post 266 may have a cylindrical body with exterior threads 268, similar to post 238, which mate with the interior threads 224 in the connector recess 222 of the shaft 214. As such, the threaded connection 224 is designed to accept the independent spherical head 260. The modular screw head 260 may be threaded onto the distal end 206 of the shaft 214 such that the screw head 260 forms the new distal end of the screw shaft 214. With the spherical head 260 attached to the inserted screw shank 214, the modular tulip 20 can then be attached and the contralateral construct 14 work can resume.

While this process introduces an additional step in the procedural workflow, the modular design still reduces the total number of screws needing to be inserted in the process, saving procedural time. The smaller diameter screw offering allows for treatment of a larger patient population as well. In the same manner as bilateral screws 100, 100A, this screw iteration can be driven and inserted by spherical head 210 or replaced by a monoaxial tulip, similar to tulip 160, depending on the implant configuration, patient anatomy, and types of constructs being created.

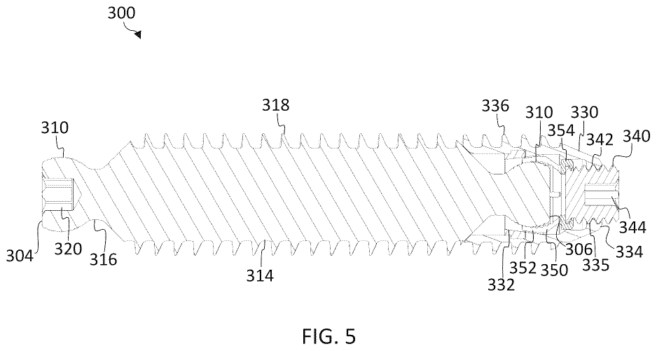

Turning now to FIG. 5, a bilateral anterior screw 300 is shown according to another embodiment. This bilateral screw 300 is similar to screw 100 but utilizes a snap-fit connection within the nose cone 330 to secure it to the screw shank 314. Like bilateral screw 100, the bilateral anterior screw 300 extends from proximal end 304 to distal end 306 and each end 304, 306 includes a modular spherical head 310 configured to couple to the respective rod or cord constructs 12, 14. Each modular screw head 310 may extend from the shaft 314 via a narrowed or reduced diameter neck 316. The proximal screw head 310 may define a drive recess 320, like drive recess 120, for engagement with an instrument to insert the screw 300.

In this embodiment, the temporary nose cone 330 may be secured to the distal screw head 310 with a set screw 340. The temporary nose cone 330 may be sized and dimensioned to cover and hide the entire distal screw head 310. The nose cone 330 may define a cavity 332, which receives the distal screw head 310 therein. The nose cone 330 includes a tapered outer body that terminates at a distal tip 334. In this embodiment, the distal tip 334 defines a recess 335 sized and dimensioned to receive the set screw 340. The recess 335 may be at least partially threaded to mate with corresponding threads 342 on the exterior of the set screw 340. The outer surface of the nose cone 330 includes one or more threads 336, which may match with the threads 118 along the shaft 114.

The temporary nose cone 330 may have an internal clamp or spherical connector 350. The spherical connector 350 may have clamping legs or deformable members 352 that grip the spherical head 310 of the bilateral screw 300. Inner surfaces of the legs or clamping members 352 may be grooved, textured, or roughened, for example, to enhance connection to the grooved spherical head 310. The locking set screw 340 is engaged with an upper portion 354 of the spherical connector 350. For example, the engagement end of the set screw 340 may include an annular protrusion configured to be received in a corresponding recess in the upper portion 345 of the connector 350. The locking set screw 340 may be rotated by engagement with an instrument in recess 344. The locking set screw 340 may be free to rotate relative to the clamp 350 allowing it to move the clamp 350 within the cavity 332 of the nose cone 330, and thereby provide a clamping force to the screw head 310.

The small set screw 340, at the nose cone's point 344, is configured to translate the spherical connector 350 within the nose cone 330. This translation compresses or relaxes the spherical connector 350 around the screw's spherical head 310, coupling the nose cone 330 to the screw 300 with distinct locked and unlocked positions. This set screw driven locking mechanism ensures the nose cone 330 does not disassociate from the screw 300 at any point during the procedure and also ensures the nose cone 330 does not rotate independently of the screw shank 314 during insertion. The nose cone set screw 340 cannot cause insertion issues as the diameter of the set screw 340 may be much smaller compared to the nose cone's overall diameter and any screw pathway preparation of the vertebra 2 may be sized to provide clearance for the nose cone set screw 340.

Alternatively, at the loss of polyaxial functionality for any tulip added, a snap-fit design without a locking set screw may also be utilized. For example, two or more flats may be created on the screw's spherical head 310 to prevent independent rotation of the nose cone 330 from the screw shank 314 once snapped into the nose cone's splay feature. The nose cone's internal connector 350 may include similar flat faces to help prevent unwanted rotation during insertion. In yet another alternative configuration, a properly sized snap-fit may be used to attach the nose cone 330 to the screw head 310 without a locking set screw. A snap-fit configuration avoids the need to lock the nose cone 330 to the shank's connection feature altogether. This eliminates the workflow step of needing to unlock the set screw 340 before nose cone 330 removal. For any of these embodiments, the screw 300 may include nose cone pre-assembly at the manufacturer to ensure thread timing between the nose cone 330 and screw shank 314 are maintained for smooth screw insertion.

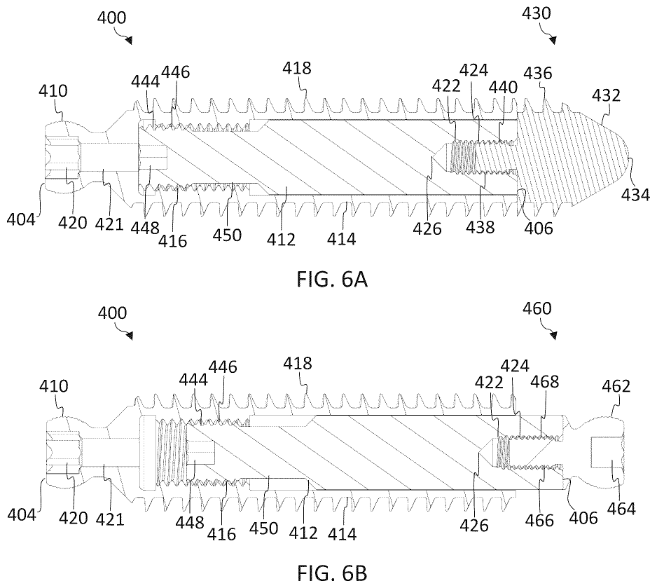

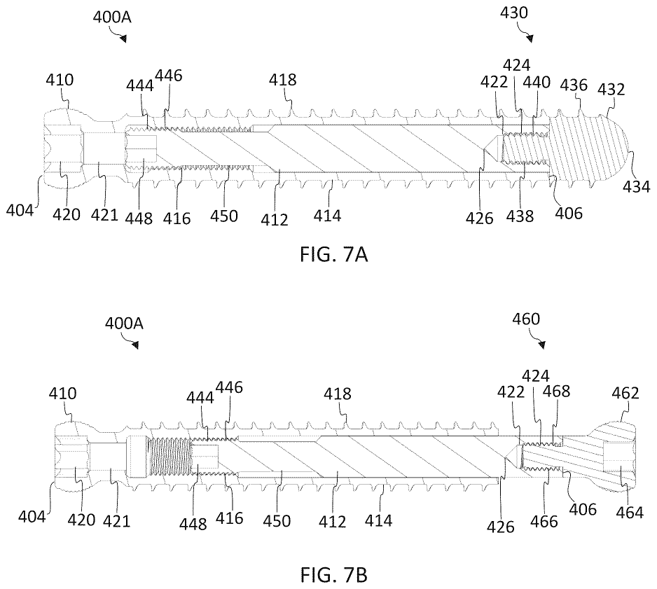

Turning now to FIGS. 6A-6B and 7A-7B, adjustable length bilateral anterior screws 400, 400A are shown according to additional embodiments. Bilateral anterior screws 400, 400A are similar to screw 200, with an interchangeable nose cone 430 or modular connector head 460, while also providing fine adjustability of the screw shank's length using a telescoping design within the shank 414. The adjustable length bilateral screws 400, 400A reduce the total number of stock-keeping units (SKUs) required to cover a wider range of patient anatomies. Additionally, surgeon's that may be less comfortable with bilateral screw designs may use a slightly shorter than intended adjustable screw and/or may make length adjustments intraoperatively. This allows the surgeon to insert the adjustable screw 400, 400A like normal but conservatively protect the contralateral anatomy from unintentional over insertion or injury. The adjustable length bilateral screws 400, 400A may be sized for both large diameter and smaller diameter screw offerings. For example, FIGS. 6A-6B depict a large diameter screw 400 for better bone purchase, and FIGS. 7A-7B depict a small diameter screw 400A, which may be better suited for small stature patients.

Similar to bilateral screw 200, bilateral anterior screws 400, 400A extend from proximal end 404 to distal end 406. The bilateral screws 400, 400A are composed of an inner shaft 412 surrounded by an outer shaft 414. The inner shaft 412 is telescopically received within the outer shaft 414 and is permitted to translate longitudinally relative to the outer shaft 414. The outer shaft 414 may have a generally hollow cylindrical body creating an inner region sized and dimensioned to receive the inner shaft 412 therein.

The proximal end 404 of the outer shaft 414 includes a modular screw head 410, for example, with a spherical shape similar to screw heads 110, 210, which is configured to receive the tulip 20 thereon. The screw head 410 defines a larger drive recess 420 for mating with a driver instrument for installing the screw 400. A central access channel 421 allows for fluid communication from the drive recess 420 to a smaller drive recess 448 in the inner shaft 412. The drive recess 420 for the outer shaft 414 may have a torx shape or star-shaped pattern, for example, or another suitably shaped tool engagement surface to insert the bone screw 400, 400A into bone. The drive recess 448 in the inner shaft 412 may be smaller and different than the larger drive recess 420. For example, the drive recess 448 of the inner shaft 412 may have a hexalobe or other suitably shaped tool engagement surface to rotate and move the inner shaft 412 relative to the outer shaft 414.

The outer shaft 414 defines one or more exterior bone threads 418 configured to engage bone. The exterior bone threads 418 may extend along the entire length of the shaft 414 or a portion thereof. Within the hollow body of the outer shaft 414, an inner region of the outer shaft 414 may include one or more interior threads 416 configured to mate with corresponding threads 446 on the inner shaft 412. The interior threads 416 may extend within a proximal portion of the outer shaft 414 such that when the inner shaft 412 is rotated, the inner shaft 412 extends from or retracts into the outer shaft 414. This interaction allows for precise linear movement of the inner shaft 412 relative to the outer shaft 414, thereby adjusting the overall length of the screw 400, 400A. At the distal end 406, the outer shaft 414 terminates bluntly as a free end.

The inner shaft 412 may also have a generally cylindrical body sized and dimensioned to move through the outer shaft 414. The inner shaft 412 includes a proximal drive portion 444 with threads 446, which engage with the internal threads 416 of the outer shaft 414. The proximal drive portion 444 may include an enlarged head portion which defines drive recess 448 to adjust the screw length. The inner shaft 412 may include a non-threaded reduced diameter section 450 separating the threaded drive portion 444 from the remainder of the inner shaft 412.

In this embodiment, the inner shaft 412 defines connector recess 422 that can be engaged, for example, with a portion of the nose cone 430 or modular head 460. The connector recess 422 may include interior threads 424, which mate with corresponding threads 440, 468 on the nose cone 430 or modular head 460, respectively. The connector recess 422 may be a closed bore or hollow cylindrical space that terminates, for example, at point 426 within the inner shaft 412.

As previously described for screw 200, the nose cone 430 or modular head 460 may be interchangeably attached to the shaft 412, in this case the inner shaft 412. The temporary nose cone 430 includes a tapered outer body 432 that terminates as a blunt distal tip 434. The outer surface of the nose cone 430 includes one or more threads 436, which may match with the threads 418 along the outer shaft 414 of the screw 400, 400A. The body 432 includes a central post 438 that extends proximally. The post 438 may have a cylindrical body with exterior threads 440 configured to mate with the interior threads 424 in the connector recess 422 of the inner shaft 412. The nose cone 430 may be rotated onto the distal end 406 of the inner shaft 412 such that the nose cone 430 temporarily forms the distal-most tip of the screw 400, 400A.

After installation and the patient is flipped over, the nose cone 430 may be removed and the modular screw head 460 affixed to the inner screw shaft 412. Similar to modular screw head 260, the modular screw head 460 includes a spherical head portion 462 with a drive recess 464. A central post 466 extends proximally from the head portion 462. The post 466 may have a cylindrical body with exterior threads 468, which mate with the interior threads 424 in the connector recess 422 of the inner shaft 412. After removal of the nose cone 430, the modular screw head 460 may be threaded onto the distal end 406 of the screw 400, 400A such that the screw head 460 forms the new distal-most end of the screw 400, 400A.

A double-featured driver may be used for initial screw insertion, followed by a smaller single-tipped driver for screw length adjustability. Once the screw's shank 414 has been positioned within the vertebral body 4 as intended, and the screw's nose cone 430 has just passed the contralateral cortical wall, a smaller driver may be introduced through the proximal end 404 of the screw 400. In another embodiment a dual-shaft instrument that allows rotation of the inner shaft and outer shaft independently may be used. The small, internal drive recess 448 allows for rotation of the screw's inner shank 412 independently of the already inserted outer shank 414. Driven by threads of a much finer pitch than the screw's outer threads 418, the surgeon may carefully adjust and expand the inserted screw's inner shank length until the ideal amount of nose cone 430 becomes exposed on the contralateral side of the vertebra 2. The first construct 12 may be completed as normal, the patient flipped, the adjustable screw's nose cone 430 removed, and the threaded modular connector 460 may be added in its place so that the second construct 14 may be completed. The internal threaded expansion mechanism enhances precision and control over the screw shank's overall length. The adjustable length option offers surgeons, especially those less experienced with the procedure, increased comfort and safety for this new procedural solution. As a result, the bilateral anterior screw method provides a time-saving and risk-reducing alternative to individually placing anterior screws on both sides of the spine.

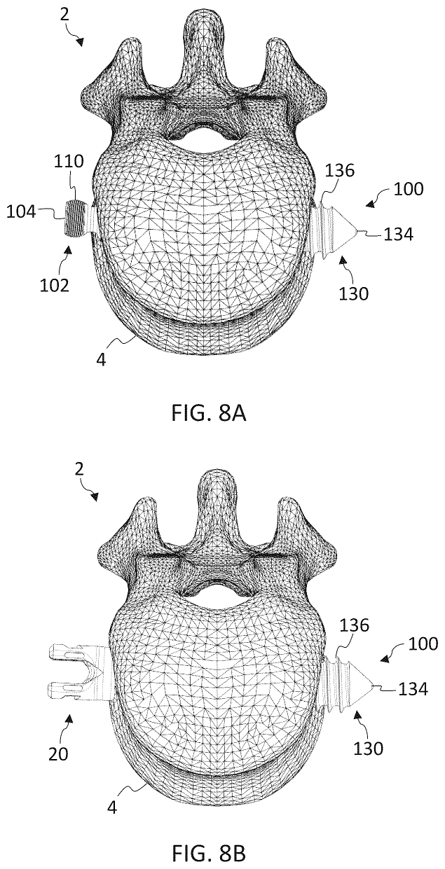

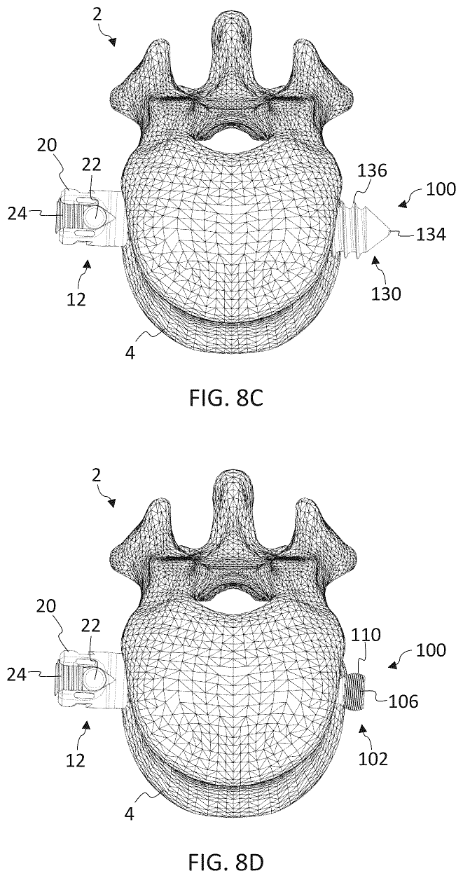

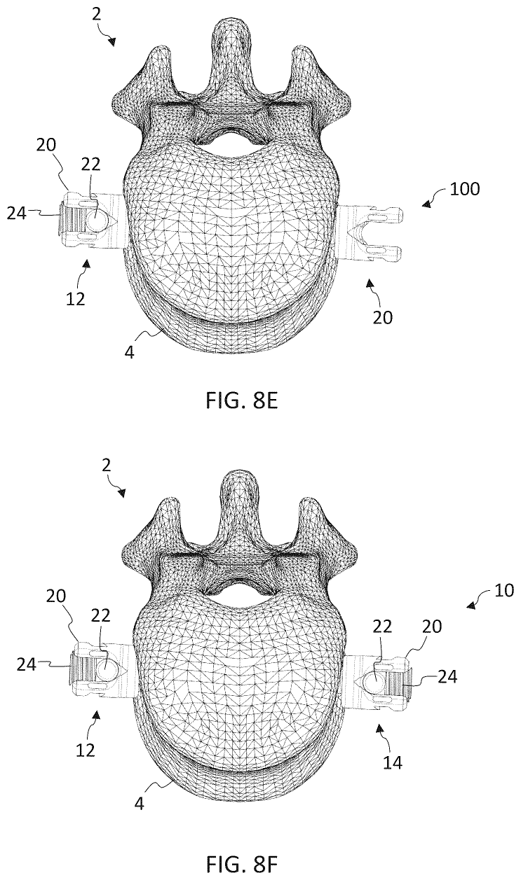

Turning now to FIGS. 8A-8F, one example of installing bilateral anterior screw system 10 is shown according to one embodiment. Although shown with reference to bilateral anterior screw 100, it will be appreciated that any of the anterior screw types may be substituted during this process. As shown in FIG. 8A, bilateral anterior screw 100 is fully inserted through vertebra 2 (e.g., thoracic vertebra T12 is shown). The temporary nose cone 130 covers and protects the distal screw head 110 during installation. In FIG. 8B, the first modular tulip 20 is placed onto the proximal screw head 110 of the screw 100 on the ipsilateral side (insertion side). In FIG. 8C, the ipsilateral construct 12 is completed with rod 22 (or cord) installed in the tulip 20 and secured via threaded locking cap 24. The ipsilateral rod 22 (or cord) may be extended to other vertebral levels, with additional bilateral anterior screw(s) or individual anterior screws from both sides of the spine, as selected by the surgeon for the desired spinal correction. In FIG. 8D, after the patient is flipped, the temporary nose cone 130 is removed to expose the distal screw head 110. In FIG. 8E, the second modular tulip 20 is placed onto the distal screw head 110 of the screw 100 on the contralateral side. In FIG. 8F, the contralateral construct 14 is completed with rod 22 (or cord) inserted into tulip 20 and secured via threaded locking cap 24. The contralateral rod 22 (or cord) may be extended to other vertebral levels, with additional bilateral anterior screw(s) or individual anterior screws from both sides of the spine, as selected by the surgeon to complete the spinal correction.

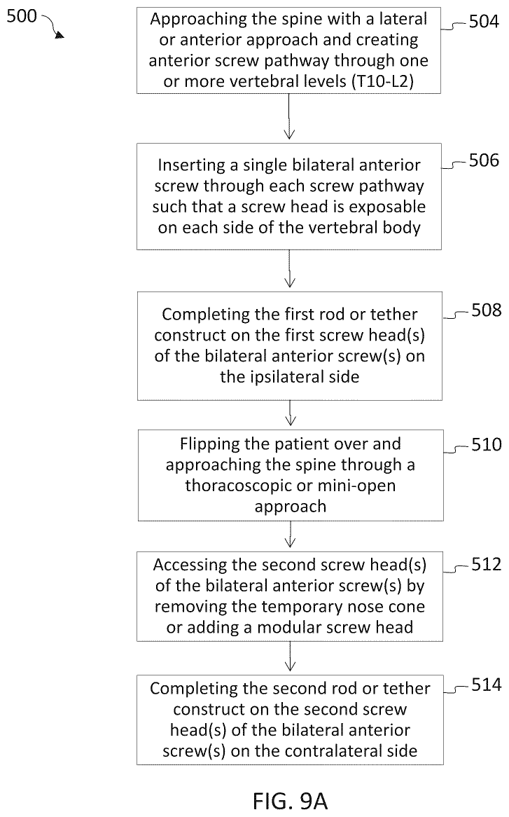

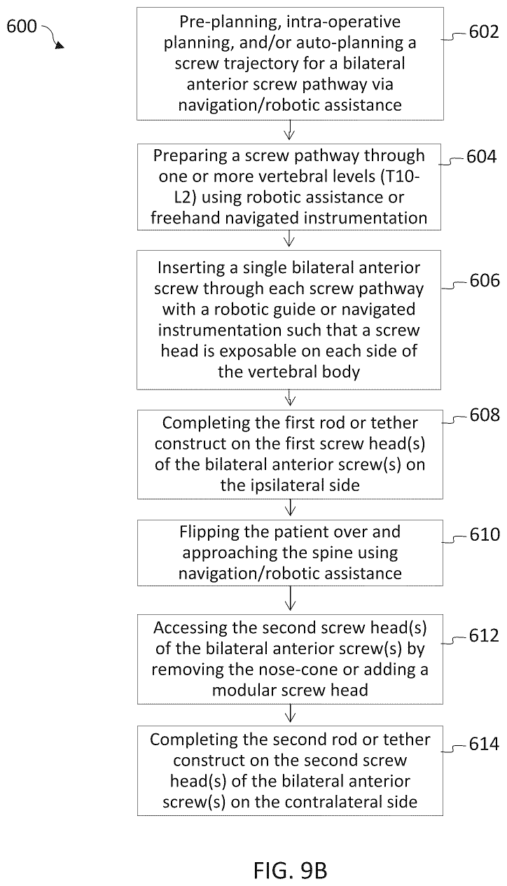

Turning now to FIGS. 9A-9B, examples of procedural workflows 500, 600 are provided in more detail. FIG. 9A describes a traditional or freehand workflow 500, which may be performed by the surgeon. FIG. 9B describes a navigated and/or robotically-assisted workflow 600, which may utilize a robot and/or navigation system to assist the surgeon with the procedure. Details of robotic and/or navigational systems can be found, for example, in U.S. Pat. Nos. 10,675,094, 9,782,229, and U.S. Patent Publication No. 2017/0239007, which are incorporated herein by reference in their entireties for all purposes.

As shown in FIG. 9A, a freehand workflow 500 may include one or more of the following steps. In step 504, the approach to the spine and pathway(s) created through the vertebra(e) may be selected to optimize screw placement. The bilateral anterior screw(s) may be inserted with a lateral or anterior approach to the spine. The bilateral screw designs allow for insertion of a single screw at each vertebral level that would typically have multiple screws inserted from each side of the vertebral body. In hybrid constructs and/or multi-curve patients, the levels needing overlapping anterior constructs may fall between vertebrae T10 and L2, for example. The bilateral screws may apply to any level in the thoracolumbar spine, however. A standard anterior approach to the spine (e.g., thoracoscopic or mini-open) may be used for bilateral screw insertion. Depending on the patient's construct needs, the applicable bilateral screw design may be selected for the appropriate level(s). Standard techniques for determining ideal screw length and trajectory may be used.

An awl and anterior probe may be used in combination with fluoroscopic imaging to determine the necessary screw length and prepare the screw pathway. Pilot drills or reamers of varying diameter may be used to further prepare the screw pathway, especially if the larger diameter screws are selected. Due to the bilateral nature of these screws, extra care may be needed when inserting these screws and advancing them past the contralateral cortical wall. Segmental vessels as well as the descending major vessels may be identified and screw trajectories strategic in avoiding these anatomical features should be planned as much as possible. Care and extra time spent preparing the screw pathway may help to ensure safe screw insertion and the additional time may be recouped by avoiding the need to insert multiple screws for that specific level.

In step 506, the bilateral screw(s) may be inserted through the respective pathway(s) such that the screw heads are exposable at each side of the vertebral body. Once an appropriate screw length is determined for a given level and the screw pathway is created, the selected screw can be inserted using a rigid screw driver that mates to the modular spherical head or directly to the monoaxial tulip. The temporary nose cone may be added to the distal end to facilitate insertion of the bilateral screw through the vertebra. Extra care may be needed as the contralateral cortex is breached. Fluoroscopic imaging and monitoring of the screw's advancement may be beneficial. In step 508, the first rod or tether construct may be completed on the ipsilateral side of the bilateral screw(s). This may include attaching a first tulip to the proximal screw head, inserting the rod or cord, and securing the assembly with a locking cap. The rest of the first construct may be completed before or after the bilateral screw(s) are implanted.

In step 510, once the first implanted side's construct and correction is complete, the patient may be flipped for implantation and assembly of the opposing construct. The contralateral side of the spine, now positioned upwards for implantation, may be accessed, for example, using a thoracoscopic or mini-open approach for creating the opposing construct. In step 512, the second screw head(s) of the bilateral anterior screw(s) may be accessed. The bilateral screw(s) nose cone(s) may be carefully removed using a customized driver that retains the nose cone and allows for the application of sufficient force to remove the nose cone from its connection with the screw shank. Alternatively, the modular screw head may substitute and replace the temporary nose cone. In step 514, the second rod or tether construct may be completed on the contralateral side of the bilateral screw(s). The appropriate modular tulip or connector may be attached to the shank's connection or spherical head. This may include attaching a second tulip to the distal screw head, inserting the rod or cord, and securing the assembly with a threaded locking cap. The rest of this side's construct may be implanted before or after the nose cone(s) are removed. The contralateral construct may then be completed as normal and any additional correction techniques may be finalized.

As shown in FIG. 9B, a navigated or robot-assisted workflow 600 may include one or more of the following steps. While the same general steps may be followed as described in the freehand workflow 500, integrated use with robotics and navigated instrumentation may greatly improve the safety and time-saving benefits of the bilateral screw systems. The anterior approach to the spine, in general, is less familiar for the majority of spine surgeons who are more comfortable with a posterior approach and technique.

In an initial step 602, the screw trajectory may be pre-planned, planned intraoperatively, and/or auto-planned by the navigated/robotic system. Leveraging the robotic platform's pre-operative planning and/or auto-planning capabilities, surgeons may better understand the screw sizes needed for specific levels as well as more strategically adjust screw trajectories to avoid areas and anatomical features of concern. Utilizing pre-operative or intra-operative scans, the surgeon may not need to spend time using fluoroscopy and an anterior probe to ascertain screw size for determining the ideal screw trajectory.

In step 604, the screw pathway may be prepared using robotic assistance or freehand navigated instrumentation. By using the robot's assistance or freehand navigated instrumentation, the surgeon can more accurately and more repeatably prepare the screw pathway through the vertebra(e), especially for the larger diameter bilateral screws. Safely and accurately preparing the screw pathway also helps to control screw insertion.

In step 606, the bilateral anterior screw(s) are inserted through each respective screw pathway with a robotic guide or navigated instrumentation such that each screw head is exposable on each side of the vertebral body. Insertion through a robotic guide may further prevent de-orbiting of the screw as its inserted through the vertebral body as well. The navigated instrumentation and robotic assistance also allow for better monitoring of the nose cone's advancement through the contralateral cortical wall. Additionally, navigated instrumentation can assist surgeons with finding and aligning with the inserted bilateral screws'nose cones more quickly and safely. This reduces the cognitive load on the surgeon and brings considerable time savings during nose cone removal as well as when introducing the modular feature or modular tulip to the already inserted shank and established trajectory.

Additionally, markings, identifiers, and/or sensors may be incorporated into the instrumentation and/or the nose cone of the screw to provide better safeguards when advancing the screw through the contralateral cortical wall. Further, the robotic software may use the patient scans to approximate bone density. Torque sensors incorporated into the instrumentation and/or force sensors placed within the nose cone itself may help notify the user when the screw is encountering cancellous vs. cortical bone as well as if the insertion trajectory has deviated significantly from the prepared screw pathway. This user feedback may help to better control nose cone advancement through the contralateral vertebral cortical wall as well as prevent misplacement of the bilateral screws.

Anterior spine surgeons may have general difficulty maintaining accurate screw trajectories during insertion of harder to reach levels especially due to the forces the skin or rib cage may be placing on the guide or port while the screw is being placed. The robotic arm may be leveraged to help resist the translational forces the patient's rib cage and skin may be exerting on any port and guide being used. This may help better maintain alignment with the planned screw trajectory and help alleviate the physical load on the surgeon during these longer, more involved bilateral procedures.

In step 608, the first rod or tether construct may be completed on the ipsilateral side of the bilateral screw(s). This may include attaching a first tulip to the proximal screw head, inserting the rod or cord, and securing the assembly with a locking cap. The rest of the first construct may be completed before or after the bilateral screw(s) are implanted.

In step 610, once the first implanted side's construct and correction is complete, the patient may be flipped for implantation and assembly of the opposing construct. The contralateral side of the spine, now positioned upwards for implantation, may be accessed, for example, using navigation or the robot system for creating the opposing construct. In step 612, the second screw head(s) of the bilateral anterior screw(s) may be accessed, for example, by removing the temporary nose cone(s) or adding modular connector(s). In step 614, the second rod or tether construct may be completed on the contralateral side of the bilateral screw(s). This may include attaching a second tulip to the distal screw head, inserting the rod or cord, and securing the assembly with a locking cap. The contralateral construct may then be completed and any additional correction techniques may be finalized.

The bilateral anterior screw systems provide a faster, safer, more reliable way of addressing anterior spinal procedures. For bilateral anterior construct patients, whether the constructs are VBT-VBT, fusion-fusion, or a combination of both, an optimized implant offering is needed. The bilateral anterior screws described herein provide the customization and modularity for handling all types and combinations of bilateral anterior constructs. The bilateral anterior screws also reduce the total number of implants needing to be placed, reduce operative time, and simplify the procedure.

The anterior approach to the spine brings with it some difficulties that surgeons may not be as familiar with or experience during a posterior approach. Because of this, some of the surgeon population may shy away from the approach altogether. The correction potential achievable from an anterior approach, however, warrants the design of new implantation, instrumentation, and new robotic/navigated techniques to make these anterior procedures safer, faster, and more repeatable to a point where surgeon adoption of anterior techniques increases. The benefits of the bilateral anterior screws described herein, as well as the time saving, safety, and user experience improvements that can be made if integrated into robotic and navigated workflows all brings a positive outlook on the adoption of anterior correction techniques in the future.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A bilateral anterior system comprising:
a bilateral anterior screw having a threaded shaft with modular connectors at each end, wherein the bilateral anterior screw is configured to extend completely through a vertebral body such that the modular connectors are exposed on both sides of the vertebral body;
an ipsilateral construct including a first rod or cord securable to the modular connector on an ipsilateral side with a first tulip and a first locking cap; and
a contralateral construct including a second rod or cord securable to the modular connector on a contralateral side with a second tulip and a second locking cap.

2. The system of claim 1, wherein the modular connectors are spherical screw heads on both sides.

3. The system of claim 1, wherein the modular connectors include a monoaxial tulip on the ipsilateral side and a spherical screw head on the contralateral side.

4. The system of claim 1, wherein the bilateral anterior screw includes a temporary nose cone configured to cover the modular connector on the contralateral side during insertion.

5. The system of claim 1, wherein the bilateral anterior screw includes a modular nose cone which is interchangeable with the modular connector on the contralateral side.

6. The system of claim 1, wherein the bilateral anterior screw includes an outer shaft and inner shaft that is permitted to translate relative to the outer shaft to extend an overall length of the bilateral anterior screw.

7. The system of claim 6, wherein the outer shaft defines one or more interior threads configured to mate with corresponding threads on a proximal drive portion of the inner shaft, and the proximal drive portion defines a drive recess reachable through an access channel through the modular connector on the ipsilateral side.

8. The system of claim 1, wherein the first and second tulips each include a tulip head having two arms defining a slot therebetween for receiving the first or second rod or cord therein, and the first and second locking caps include threaded set screws configured to secure the first or second rod or cord in the respective tulip heads.

9. A method for treating a spinal deformity of a patient, the method comprising:
accessing a vertebral body of a vertebra through an anterior or lateral approach;
inserting a bilateral anterior screw completely through the vertebral body such that modular connectors are exposable on an ipsilateral side and a contralateral side of the vertebral body;
attaching and securing a first rod or cord onto the modular connector on the ipsilateral side to form an ipsilateral construct;
flipping the patient over and accessing the vertebral body on the contralateral side;
exposing the modular connector on the contralateral side by: (a) removing a temporary nose cone to reveal the modular connector hidden by the temporary nose cone, or (b) replacing a modular nose cone with the modular connector; and
attaching and securing a second rod or cord onto the modular connector on the contralateral side to form a contralateral construct.

10. The method of claim 9, wherein the bilateral anterior screw traverses generally laterally across the vertebral body and spans an entire width of the vertebral body.

11. The method of claim 9, wherein the bilateral anterior screw passes through a first cortical wall on the ipsilateral side, through cancellous bone, and through a second cortical wall on the contralateral side of the vertebral body.

12. The method of claim 9, wherein prior to flipping the patient over, extending an overall length of the bilateral anterior screw by rotating an inner shaft relative to an outer shaft of the bilateral anterior screw.

13. The method of claim 9, wherein the ipsilateral and contralateral constructs are extended and secured to additional vertebrae.

14. The method of claim 9, wherein prior to accessing the vertebral body, pre-planning or auto-planning a screw trajectory for the bilateral anterior screw via navigation and/or robotic assistance.

15. The method of claim 9, wherein the ipsilateral and contralateral constructs are vertebral body tethering and vertebral body tethering, rod fusion and rod fusion, or a combination of both.

16. A bilateral anterior screw assembly comprising:
a bilateral anterior screw having a threaded shaft with a proximal modular connector at one end and a distal modular connector at an opposite end; and
a removable nose cone configured to temporarily cover the distal modular connector for insertion of the bilateral anterior screw into bone,
wherein the bilateral anterior screw is configured to extend completely through a vertebral body, and after implantation, the nose cone is removable to expose the distal modular connector.

17. The assembly of claim 16, wherein the distal modular connector defines a connector recess with interior threads that mate with a post having corresponding threads within the nose cone.

18. The assembly of claim 16, wherein the proximal and distal modular connectors are each spherical screw heads.

19. The assembly of claim 18, wherein the nose cone defines a cavity, which receives the spherical screw head of the distal modular connector, and a tapered outer body that terminates as a blunt distal tip.

20. The assembly of claim 16, wherein the nose cone includes one or more threads, which are timed with bone threads along the threaded shaft.
